(19) United States
(12) Patent Application Publication
(10) Pub. No.: US 2024/0221962 A1
(43) Pub. Date: Jul. 4, 2024

(54) METAVERSE CARE AND RETAIL EXPERIENCE

(71) Applicant: T-MOBILE INNOVATIONS LLC, Overland Park, KS (US)

(72) Inventors: Praveen Chakravarthy SATTARU, Lynnwood, WA (US); Rajesh Kalathil NARAYANAN, Sammamish, WA (US)

(21) Appl. No.: 18/149,390

(22) Filed: Jan. 3, 2023

(51) Int. Cl. G16H 80/00; G06F 3/01

(52) U.S. Cl. CPC G16H 80/00; G06F 3/011

(57) ABSTRACT

According to aspects herein, methods and systems for management of a customer care agent application operated within a metaverse. A user may access the customer care agent application using a shortcut within the metaverse. Such a shortcut will allow the user to begin to interact with an agent avatar. The agent avatar will then attempt to identify a concern or reason the user has begun interacting with the customer care agent application. If the system itself is unable to identify the concern or reason, a human interaction will occur. Solutions will be found within a personalized database or using a human intervention. In all cases, the agent avatar will appear as if not controlled or directed by more than one individual or computer processor. All interactions with the user by the agent avatar will appear seamless and personalized.

### BACKGROUND

In the metaverse, people may rent or buy virtual store space, conference rooms, social environments, virtual theater, game room, parks, or similar gathering spaces. These spaces will likely want to provide customer service options within these virtual locations and require large numbers of customer service representatives needed in the real world to provide a personalized customer care experience. The ideal customer service interaction is a highly personalized care agent that is the same agent each time a user interacts with it. In an ideal world the customer care agent would learn everything possible about the user in order to provide a highly personalized customer service interaction.

### SUMMARY

A high-level overview of various aspects of the present technology is provided in this section to introduce a selection of concepts that are further described below in the detailed description section of this disclosure. This summary is not intended to identify key or essential features of the claimed subject matter, nor is it intended to be used as an aid in isolation to determine the scope of the claimed subject matter.

The agent avatar will then attempt to identify a concern of reason the user has begun interacting with the customer care agent application. If the system itself is unable to identify the concern or reason, a human interaction will occur. Solutions will be found within a personalized database or using a human intervention. In all cases, the agent avatar will appear as if not controlled or directed by more than one individual or computer processor. All interactions with the user by the agent avatar will appear seamless and personalized.

### BRIEF DESCRIPTION OF THE SEVERAL VIEWS OF THE DRAWINGS

Implementations of the present disclosure are described in detail below with reference to the attached drawing figures, wherein:

FIG. 1 depicts a computing environment suitable for use in implementations of the present disclosure, in accordance with aspects herein;

FIG. 2 depicts a diagram of an exemplary network environment in which implementations of the present disclosure may be employed, in accordance with aspects herein; and

FIG. 3 is a flowchart of a method in accordance with aspects herein.

### DETAILED DESCRIPTION

The subject matter of embodiments of the invention is described with specificity herein to meet statutory requirements. However, the description itself is not intended to limit the scope of this patent. Rather, the inventors have contemplated that the claimed subject matter might be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

Throughout this disclosure, several acronyms and shorthand notations are employed to aid the understanding of certain concepts pertaining to the associated system and services. These acronyms and shorthand notations are intended to help provide an easy methodology of communicating the ideas expressed herein and are not meant to limit the scope of embodiments described in the present disclosure. Various technical terms are used throughout this description. An illustrative resource that fleshes out various aspects of these terms can be found in Newton's Telecom Dictionary, 32nd Edition (2022).

Embodiments of the present technology may be embodied as, among other things, a method, system, or computer-program product. Accordingly, the embodiments may take the form of a hardware embodiment, or an embodiment combining software and hardware. An embodiment takes the form of a computer-program product that includes computer-usable instructions embodied on one or more computer-readable media.

Computer-readable media include both volatile and nonvolatile media, removable and non-removable media, and contemplate media readable by a database, a switch, and various other network devices. Network switches, routers, and related components are conventional in nature, as are means of communicating with the same. By way of example, and not limitation, computer-readable media comprise computer-storage media and communications media.

Computer-storage media, or machine-readable media, include media implemented in any method or technology for storing information. Examples of stored information include computer-useable instructions, data structures, program modules, and other data representations. Computer-storage media include, but are not limited to RAM, ROM, EEPROM, flash memory or other memory technology, CD-ROM, digital versatile discs (DVD), holographic media or other optical disc storage, magnetic cassettes, magnetic tape, magnetic disk storage, and other magnetic storage devices. These memory components can store data momentarily, temporarily, or permanently.

Communications media typically store computer-useable instructions—including data structures and program modules—in a modulated data signal. The term “modulated data signal” refers to a propagated signal that has one or more of its characteristics set or changed to encode information in the signal. Communications media include any information-delivery media. By way of example but not limitation, communications media include wired media, such as a wired network or direct-wired connection, and wireless media such as acoustic, infrared, radio, microwave, spread-spectrum, and other wireless media technologies. Combinations of the above are included within the scope of computer-readable media.

By way of background, a traditional telecommunications network employs a plurality of base stations (i.e., access point, node, cell sites, cell towers) to provide network coverage. The base stations are employed to broadcast and transmit transmissions to user devices of the telecommunications network. An access point may be considered to be a portion of a base station that may comprise an antenna, a radio, and/or a controller. In aspects, an access point is defined by its ability to communicate with a user equipment (UE), such as a wireless communication device (WCD), according to a single protocol (e.g., 3G, 4G, LTE, 5G, 6G, and the like); however, in other aspects, a single access point may communicate with a UE according to multiple protocols. As used herein, a base station may comprise one access point or more than one access point. Factors that can affect the telecommunications transmission include, e.g., location and size of the base stations, and frequency of the transmission, among other factors. The base stations are employed to broadcast and transmit to user devices of the telecommunications network. Traditionally, the base station establishes uplink (or downlink) transmission with a mobile handset over a single frequency that is exclusive to that particular uplink connection (e.g., an LTE connection with an EnodeB). In this regard, typically only one active uplink connection can occur per frequency. The base station may include one or more sectors served by individual transmitting/receiving components associated with the base station (e.g., antenna arrays controlled by an EnodeB). These transmitting/receiving components together form a multi-sector broadcast arc for communication with mobile handsets linked to the base station.

As used herein, UE (also referenced herein as a user device or a wireless communication device) can include any device employed by an end-user to communicate with a wireless telecommunications network. A UE can include a mobile device, a mobile broadband adapter, a fixed location or temporarily fixed location device, or any other communications device employed to communicate with the wireless telecommunications network. For an illustrative example, a UE can include cell phones, smartphones, tablets, laptops, small cell network devices (such as micro cell, pico cell, femto cell, or similar devices), and so forth. Further, a UE can include a sensor or set of sensors coupled with any other communications device employed to communicate with the wireless telecommunications network; such as, but not limited to, a camera, a weather sensor (such as a rain gauge, pressure sensor, thermometer, hygrometer, and so on), a motion detector, or any other sensor or combination of sensors. A UE, as one of ordinary skill in the art may appreciate, generally includes one or more antennas coupled to a radio for exchanging (e.g., transmitting and receiving) transmissions with a nearby base station or access point.

According to aspects herein, methods and systems for managing a customer care agent operated within a virtual environment. The method begins with receiving from a user device a request for a user to access the care agent application. A user's identity is authenticated. The method then causes the personalized care agent to interact with the user and identify a first user concern. The method access a personalized care database associated with the user to identify a solution to the first user concern. Once the concern is identified, the method then causes the personalized care agent to communicate the solution to the user.

The present disclosure also provides a method for capacity management of virtual space in a network. The method begins with receiving from a user device a request for a user to access the care agent application. A user's identity is authenticated. The method then causes the personalized care agent to interact with the user and identify a first user concern. The method access a personalized care database associated with the user to identify a solution to the first user concern. Once the concern is identified, the method then causes the personalized care agent to communicate the solution to the user.

In addition, the present disclosure provides a non-transitory computer storage media storing computer-usable instructions that, that when used by the processor, cause the processor to perform the following operations: receiving from a user device a request for a user to access the care agent application. A user's identity is authenticated. The method then causes the personalized care agent to interact with the user and identify a first user concern. The method access a personalized care database associated with the user to identify a solution to the first user concern. Once the concern is identified, the method then causes the personalized care agent to communicate the solution to the user.

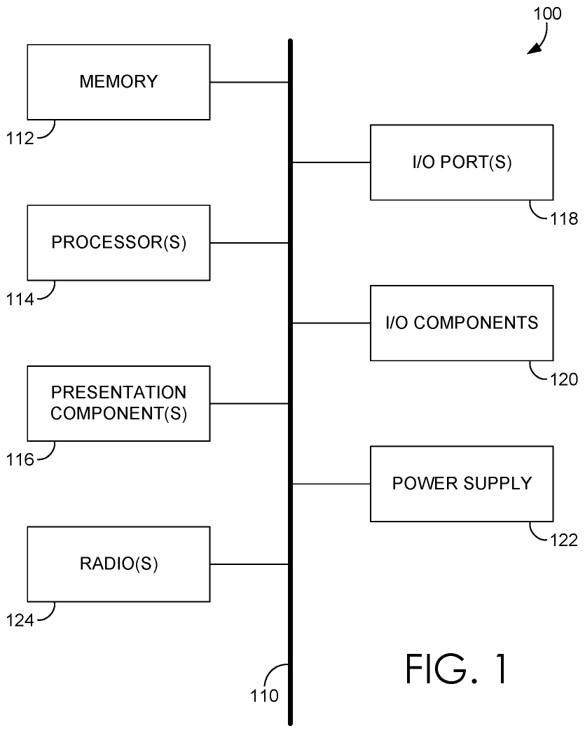

FIG. 1 depicts a computing environment suitable for use in implementations of the present disclosure. In particular, the exemplary computer environment is shown and designated generally as computing device 100. Computing device 100 is but one example of a suitable computing environment and is not intended to suggest any limitation as to the scope of use or functionality of the invention. Neither should computing device 100 be interpreted as having any dependency or requirement relating to any one or combination of components illustrated. In aspects, the computing device 100 may be a UE, or other user device, capable of two-way wireless communications with an access point. Some non-limiting examples of the computing device 100 include a cell phone, tablet, pager, personal electronic device, wearable electronic device, activity tracker, desktop computer, laptop, PC, and the like.

The implementations of the present disclosure may be described in the general context of computer code or machine-useable instructions, including computer-executable instructions such as program components, being executed by a computer or other machine, such as a personal data assistant or other handheld device. Generally, program components, including routines, programs, objects, components, data structures, and the like, refer to code that performs particular tasks or implements particular abstract data types. Implementations of the present disclosure may be practiced in a variety of system configurations, including handheld devices, consumer electronics, general-purpose computers, specialty computing devices, etc. Implementations of the present disclosure may also be practiced in distributed computing environments where tasks are performed by remote-processing devices that are linked through a communications network.

With continued reference to FIG. 1, computing device 100 includes bus that directly or indirectly couples the following devices: memory 112, one or more processors 114, one or more presentation components 116, input/output (I/O) ports 118, I/O components 120, radio 116, transmitter 118, and power supply 114. Bus represents what may be one or more busses (such as an address bus, data bus, or combination thereof). Although the devices of FIG. 1 are shown with lines for the sake of clarity, in reality, delineating various components is not so clear, and metaphorically, the lines would more accurately be grey and fuzzy. For example, one may consider a presentation component such as a display device to be one of I/O components 120. Also, processors, such as one or more processors 114, have memory. The present disclosure hereof recognizes that such is the nature of the art, and reiterates that FIG. 1 is merely illustrative of an exemplary computing environment that can be used in connection with one or more implementations of the present disclosure. Distinction is not made between such categories as “workstation,” “server,” “laptop,” “handheld device,” etc., as all are contemplated within the scope of FIG. 1 and refer to “computer” or “computing device.”

Computing device 100 typically includes a variety of computer-readable media. Computer-readable media can be any available media that can be accessed by computing device 100 and includes both volatile and nonvolatile media, removable and non-removable media. By way of example, and not limitation, computer-readable media may comprise computer storage media and communication media. Computer storage media includes both volatile and nonvolatile, removable and non-removable media implemented in any method or technology for storage of information such as computer-readable instructions, data structures, program modules or other data. Computer storage media includes RAM, ROM, EEPROM, flash memory or other memory technology, CD-ROM, digital versatile disks (DVD) or other optical disk storage, magnetic cassettes, magnetic tape, magnetic disk storage or other magnetic storage devices. Computer storage media does not comprise a propagated data signal.

Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer-readable media.

Memory 112 includes computer-storage media in the form of volatile and/or nonvolatile memory. Memory 112 may be removable, non-removable, or a combination thereof. Exemplary memory includes solid-state memory, hard drives, optical-disc drives, etc. Computing device 100 includes one or more processors 114 that read data from various entities such as bus, memory 112 or I/O components 120. One or more presentation components 116 present data indications to a person or other device. Exemplary one or more presentation components 116 include a display device, speaker, printing component, vibrating component, etc. I/O ports 118 allow computing device 100 to be logically coupled to other devices including I/O components 120, some of which may be built into computing device 100. Illustrative I/O components 120 include a microphone, joystick, game pad, satellite dish, scanner, printer, wireless device, etc.

The radio 116 represents one or more radios that facilitate communication with a wireless telecommunications network. While a single radio 116 is shown in FIG. 1, it is contemplated that there may be more than one radio 116 coupled to the bus. In aspects, the radio 116 utilizes a transmitter 118 to communicate with the wireless telecommunications network. It is expressly conceived that a computing device with more than one radio 116 could facilitate communication with the wireless telecommunications network via both the first transmitter 118 and an additional transmitters (e.g. a second transmitter). Illustrative wireless telecommunications technologies include CDMA, GPRS, TDMA, GSM, and the like. The radio 116 may additionally or alternatively facilitate other types of wireless communications including Wi-Fi, WiMAX, LTE, 3G, 4G, LTE, 5G, NR, VOLTE, or other VoIP communications. As can be appreciated, in various embodiments, radio 116 can be configured to support multiple technologies and/or multiple radios can be utilized to support multiple technologies. A wireless telecommunications network might include an array of devices, which are not shown so as to not obscure more relevant aspects of the invention. Components such as a base station, a communications tower, or even access points (as well as other components) can provide wireless connectivity in some embodiments.

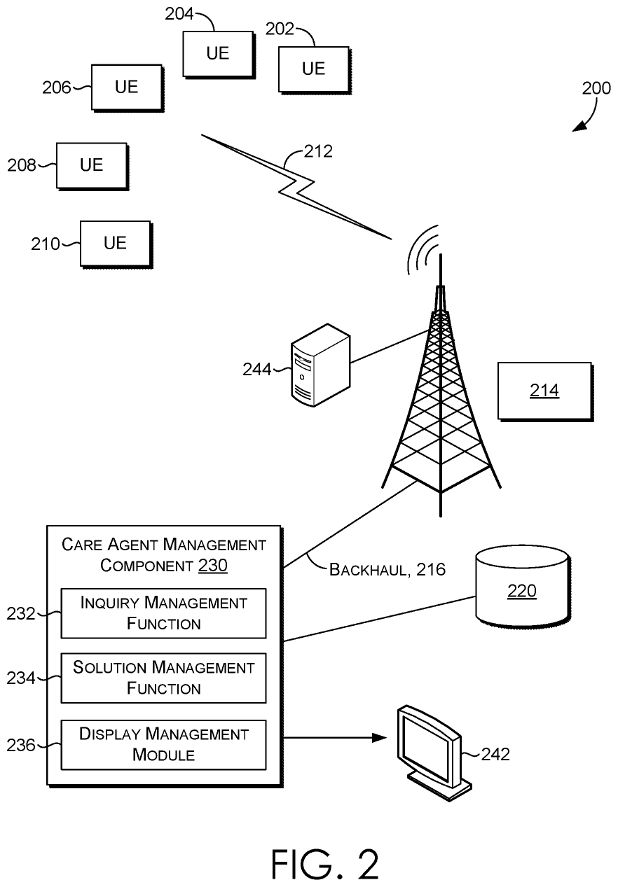

FIG. 2 depicts a diagram of an exemplary network environment in which implementations of the present disclosure may be employed. Such a network environment is illustrated and designated generally as network environment 200. Network environment 200 is not to be interpreted as having any dependency or requirement relating to any one or combination of components illustrated.

Network environment 200 includes user devices (UE) 202, 204, 206, 208, and 210, access point 214 (which may be a cell site, base station, or the like), and one or more communication channels 212. In network environment 200, user devices may take on a variety of forms, such as a personal computer (PC), a user device, a smart phone, a smart watch, a laptop computer, a mobile phone, a mobile device, a tablet computer, a wearable computer, a personal digital assistant (PDA), a server, a CD player, an MP3 player, a global positioning system (GPS) device, a video player, a handheld communications device, a workstation, a router, a hotspot, and any combination of these delineated devices, or any other device (such as the computing device 100) that communicates via wireless communications with the access point 214 in order to interact with a public or private network.

In some aspects, each of the UEs 202, 204, 206, 208, and 210 may correspond to computing device 100 in FIG. 1. Thus, a UE can include, for example, a display(s), a power source(s) (e.g., a battery), a data store(s), a speaker(s), memory, a buffer(s), a radio(s) and the like. In some implementations, for example, a UEs 202, 204, 206, 208, and 210 comprise a wireless or mobile device with which a wireless telecommunication network(s) can be utilized for communication (e.g., voice and/or data communication). In this regard, the user device can be any mobile computing device that communicates by way of a wireless network, for example, a 3G, 4G, 5G, LTE, CDMA, or any other type of network.

In some cases, UEs 202, 204, 206, 208, and 210 in network environment 200 can optionally utilize one or more communication channels 212 to communicate with other computing devices (e.g., a mobile device(s), a server(s), a personal computer(s), etc.) through access point 214. The network environment 200 may be comprised of a telecommunications network(s), or a portion thereof. A telecommunications network might include an array of devices or components (e.g., one or more base stations), some of which are not shown. Those devices or components may form network environments similar to what is shown in FIG. 2, and may also perform methods in accordance with the present disclosure. Components such as terminals, links, and nodes (as well as other components) can provide connectivity in various implementations. Network environment 200 can include multiple networks, as well as being a network of networks, but is shown in more simple form so as to not obscure other aspects of the present disclosure.

The one or more communication channels 212 can be part of a telecommunication network that connects subscribers to their immediate telecommunications service provider (i.e., home network carrier). In some instances, the one or more communication channels 212 can be associated with a telecommunications provider that provides services (e.g., 3G network, 4G network, LTE network, 5G network, and the like) to user devices, such as UEs 202, 204, 206, 208, and 210. For example, the one or more communication channels may provide voice, SMS, and/or data services to UEs 202, 204, 206, 208, and 210, or corresponding users that are registered or subscribed to utilize the services provided by the telecommunications service provider. The one or more communication channels 212 can comprise, for example, a 1× circuit voice, a 3G network (e.g., CDMA, CDMA2000, WCDMA, GSM, UMTS), a 4G network (WiMAX, LTE, HSDPA), or a 5G network.

In some implementations, access point 214 is configured to communicate with a UE, such as UEs 202, 204, 206, 208, and 210, that are located within the geographic area, or cell, covered by radio antennas of access point 214. Access point 214 may include one or more base stations, base transmitter stations, radios, antennas, antenna arrays, power amplifiers, transmitters/receivers, digital signal processors, control electronics, GPS equipment, and the like. In particular, access point 214 may selectively communicate with the user devices using dynamic beamforming.

As shown, access point 214 is in communication with a care agent management component 230 and at least a network database 220 via a backhaul channel 216. The access point may also host a server 244 that stores applications and metaverse content that are frequently requested by users in the vicinity of access point 214. As the UEs 202, 204, 206, 208, and 210 collect individual status data, the status data can be automatically communicated by each of the UEs 202, 204, 206, 208, and 210 to the access point 214. Access point 214 may store the data communicated by the UEs 202, 204, 206, 208, and 210 at a network database 220. Alternatively, the access point 214 may automatically retrieve the personal or user data from the UEs 202, 204, 206, 208, and 210, and similarly store the data in the network database 220. The data may be communicated or retrieved and stored periodically within a predetermined time interval which may be in seconds, minutes, hours, days, months, years, and the like. With the incoming of new data, the network database 220 may be refreshed with the new data every time, or within a predetermined time threshold so as to keep the status data stored in the network database 220 current. For example, the data may be received at or retrieved by the access point 214 every 10 minutes and the data stored at the network database 220 may be kept current for 30 days, which means that status data that is older than 30 days would be replaced by newer status data at 10 minute intervals. As described above, the status data collected by the UEs 202, 204, 206, 208, and 210 can include, for example, service state status, the respective UE's current geographic location, a current time, a strength of the wireless signal, available networks, user information, user preference information, customer information (including among other things; customer preferences, account login information, customer profile information, and the like), UE device information, payment information, collections information, historical information, demographics, and geographical location and the like. In one embodiment, the historical information includes prior interactions the user has had with the care agent application or any other interactions that may be stored within the database. Historical search information and historical purchases may be stored therein. The network database 220 may be user specific and store information related to the user of each UE 202-210. Each user may have a separate account and profile stored with the network database 220. The profile will have information and preferences related to the care agent and the care agent avatar. Such information may be the avatar's appearance, language, and method of speaking, among other things.

The care agent management component 230 comprises various engines including an inquiry management function 232, a solution management function 234, and a display management module 236 may be stored at the network database 220. Although the care agent management component 230 is shown as a single component comprising the inquiry management function 232, a solution management function 234, and a display management module 236 it is also contemplated that each of the inquiry management function 232, a solution management function 234, and a display management module 236 may reside at different locations, be its own separate entity, and the like, within the home network carrier system.

The care agent management component 230 allows a mobile network operator to host an application server, such as server 244 in the mobile operator's location. This brings the server 244 closer to the UEs, such as UEs 202, 204, 206, 208, and 210, reducing latency for the UEs. The server 244 may operate using the care agent management component 230 an application which hosts a virtual care agent within a metaverse. The Care agent management component 230 operates the application within the metaverse, hosting a virtual shop or retail space where the user may virtually enter within the metaverse. The care agent management component 230 may also operate the application on multiple metaverse platforms simultaneously. The user may be able to access the care agent application by way of a shortcut within the user's metaverse landscape or system. For example, a user may be operating a metaverse application within UE 202. The user may click on a shortcut within the metaverse landscape which prompts the user to input user information or to sign in to the care agent application.

The care agent management component 230 receives a request from the one or more of the UE's 202-210 to access the care agent application hosted on the application server 244 within the metaverse. For example, the user of UE 202 may request to sign into the care agent application within a T-Mobile based metaverse by entering a virtual store or space which represents the care agent application hosted by the application server 244. In other examples, the metaverse is operated by Meta, Microsoft, Apple, or any other metaverse hosting system.

The care agent management component 230 operates the care agent application hosted by the application server 244. The care agent application operates within the metaverse world having an application program interface (API) within the metaverse. For example, the care agent application may have a space purchased or rented within the metaverse where the user may go and enter the API associated with the care agent application. The API operated by the care agent management component 230 may have a space where the user may engage with a care agent avatar. The care agent may present as an avatar which the user of the UE 202 may select and provide preferences for the avatar. For example, the user may select the avatar's appearance, preferred language, gender, hair color, age, tone of voice, posture, and other relevant preferences which may apply to how the avatar looks, speaks, and acts.

The inquiry management function 232 is provided for the gathering of information from the user of the one or more UEs 202-210. The user of a UE 202 is signed in or the user account is authenticated by the inquiry management function 232 using user credentials, facial recognition, fingerprint recognition, or other methods of authenticating a user. The user management function 232 submits an inquiry to the user by way of the API and UE for a user input or user credential which may compared against information associated with the user and is stored within the network database 220.

Once the user is authenticated by the inquiry management function 232, the care agent avatar for the user appears within the API and begins to inquire the user. The care agent application may user the care agent avatar to gather information from the user about the reason they are at the care agent application. The care agent application may use a series of questions to gather information about the concern, question, or reason the user is attempting to interact with the care agent avatar in the care agent application. The inquiry management function 232 may have protocols built in to the system which prompts the care agent avatar to probe the user with particular questions. The inquiry management function 232 may also use machine learning algorithms and user information stored within the network database to identify and learn what the user may be inquiring about. For example, the user may regularly ask about their bill in the middle of the month. The inquiry management function 232 may identify using machine learning and user information that the user is inquiring at the middle of the month.

In one embodiment, the inquiry management function 232 may use the location of the user's device to identify common questions related to that location. For example, the user device may be in a location experiencing an environmental disaster, the inquiry management function 232 may then initially prompt the care agent avatar to ask if the user is asking about the environmental disaster. Additional information and machine learning may be done to further improve the inquiry management function 232 to identify the question or concern the user has. In an additional embodiment, the user may be desiring to purchase an item or service from the customer care application. Thus, the concern or question identified will be an item or service desired.

In an additional embodiment, the inquiry management function 232 may not be able to identify the concern or question. A team of experts then provides additional help and instructions to the care agent avatar. The team of experts will provide voice or typed instructions for the care agent avatar to convey to the user. In one example, the team of experts consists of at least one individual who may respond to the inquiry or instruct the care agent avatar to respond in a particular fashion. As such, the care agent avatar will appear to be the same voice, appearance, and intonation as before but will be now operated by a human rather than the application server 244. Thus, the customer or user experiences only a one on one relationship with their care agent avatar and not multiple care agents. The user may, in some embodiments, confirm to the care agent avatar that the inquiry management function 232 has identified the correct concern or question.

The solution management function 234 is primarily responsible for identifying of a solution to the concern identified by the inquiry management function 232. Once the concern or question is identified by the inquiry management function 232, the solution management function 234 identifies a solution to the concern or question. The solution management function may search the network database 220 in its entirety for a solution for the user. The network database 220, as explained above, holds information specific to the user of each UE. For example, the user may have stored within the network database 220 information related to billing, personal information, or other items which the user may inquire about. Additionally, the solution management function 234 may use machine learning algorithms to learn solutions to common, recurring, or complex questions or concerns. In the event the solution management function 234 is unable to identify a solution, a member of the team of experts may provide instructions or a solution. For example, if the inquiry management function 232 identifies that the user is requesting a refund of $100, the solution management function 234 may not be able to provide that solution. As such, the team of experts may authorize the refund or instruct the care agent avatar to respond by telling the user that only $90 may be refunded. Thus, the solution management function 234 first uses the server 244 to search the database for a solution and then relies of the team of experts to provide additional support or answers if needed.

The display management module 236 receives all instructions within the care agent management component 230 to cause to be displayed on the UEs 202-210 the environment and care agent avatar associated with each user. The user of each UE 202-210 may select the environment they wish to interact with the care agent avatar in. For example, the care agent avatar may be in a room with chairs, in a restaurant, on a beach, or any other location or setting. The care agent avatar is also selected by the user to have a particular appearance, clothing, speech, language, gender, and other preferences related to the avatar.

The inquiry management function 232 instructs the display management module 236 to cause the display and speech of the care agent avatar to convey the instructions and questions as described above. The display management module 236 may also identify emotions that can be or should be conveyed by the care agent avatar. For example, the customer may be asking about a promotion and the display management module 236 can then identify that the care agent avatar should convey excited emotions to properly sell the promotion. Additionally, the display management module may use machine learning to identify proper emotions and actions for the care agent avatar to have. The machine learning model may learn certain emotions displayed by the care agent avatar have positive or negative reactions by the user.

The solution management function 234 also instructs the display management module to cause the display management module 236 to cause the display and speech of the care agent avatar to convey the instructions and solutions described above. The display management module 236 may also identify emotions that can be or should be conveyed by the care agent avatar. For example, the solution identified by the solution management function 234 may be associated with a negative situation. The display management module 236 can then identify that the care agent avatar should convey somber emotions to properly address the negative situation. Additionally, the display management module may use machine learning to identify proper emotions and actions for the care agent avatar to have. The machine learning model may learn certain emotions displayed by the care agent avatar have positive or negative reactions by the user.

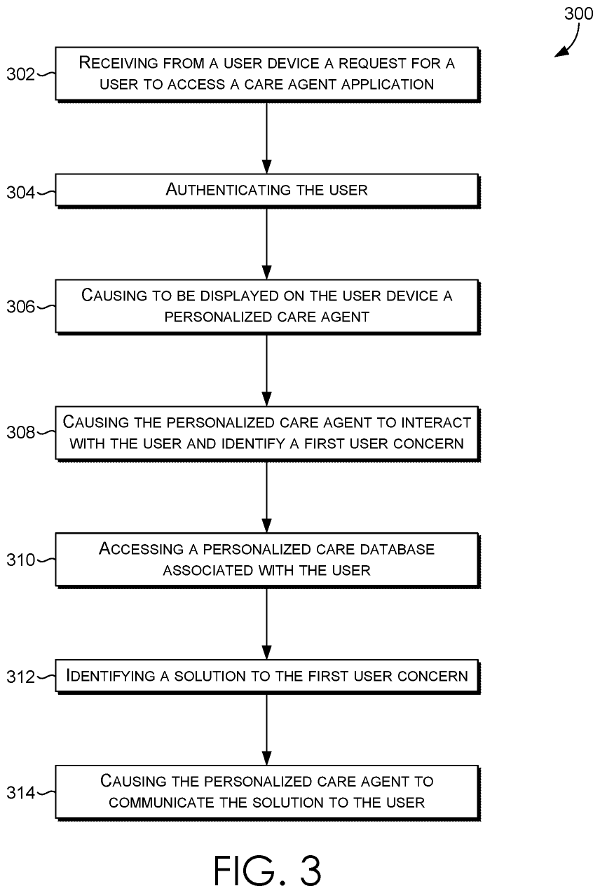

FIG. 3 is a flowchart of a method for managing a care agent avatar within a metaverse environment. The method 300 begins in step 302 with a user of a UE such as UEs 202-210 requesting access to the care agent application. The user may enter the metaverse of their choosing such as a T-Mobile® hosted metaverse. Once within that metaverse, the user may find a shortcut or a location associated with the care agent application within the metaverse. The user then selects or requests authorization to enter the care agent application associated with the user. For example, the user may request to access the care agent avatar associated with the user and located within the care agent application. The request to enter or access the care agent application requires the user to enter or submit identifying credentials associated with the user.

At step 304, the care agent application verifies or authenticates that the user credentials are valid. The care agent application authenticates the user based on stored identifying information stored within the database such as network database 220. Step 304 may use identifying credentials such as user id and password, facial recognition, fingerprint identification, or the like. Continuing with step 306, the care agent application causes to be displayed within the metaverse environment, a personalized care agent avatar. The personalized avatar may be personalized by the user such that it represents an individual they wish to be their care agent. Such personalization preferences are stored in a user specific portion of a network database. The avatar may be personalized such that it looks a particular way with particular clothing, hair, looks, gender, speech patterns, language, overall looks, demeanor, and many other particular portions of the avatar. By personalizing the avatar, each time the user accesses the care agent application within the metaverse, the care agent interacting with the user will be the same avatar and act the same. Thus, the user may get a one on one personalized care experience within the metaverse care agent application.

Now looking at step 308, the system causes the personalized care agent avatar to interact with the authorized user. The interactions by the personalized care agent avatar is directed to identifying a user concern or question to be answered or solved by the care agent application. The interactions may be directed by a script stored on a database to identify user concerns or questions. Such scripts may have prompting questions and reciprocal questions based on the user's responses. Additionally, the personalized care application may use machine learning to develop new questions to identify common or uncommon questions.

In one embodiment, the application may learn what specific questions the user may ask so that the application may identify such a question quicker than using the canned scripts. Additionally, the application may use geographic or event information to predict questions or concerns that a user may have. Moreover, if the care agent system is unable to identify a concern or question, an expert may provide additional instructions to the care agent of what questions to ask or how the care agent should interact with the user to identify the concern or question. For example, if the care agent is unable to identify a specific question, the human expert may provide a question for the avatar to ask the user such that the concern may be properly identified. In once example, the system may alert the human expert that help is needed once a concern is not identified after a set number of interactions with the user.

At step 310, a personalized care database is accessed. The personalized care database is associated with the user and contains information related to the user. Such information may be profile information, user preferences, payment information, and the like. By accessing the personal care database, the system may tailor a response to the identified question to the specific user. Now with step 312, a solution may be identified to the concern or question posed by the user. By accessing the database, the solution may be user specific, such as to address the user's concern directly. The solution may include payment, billing, or other personal information related to the user's account. Other personal solutions may be identified. In an additional embodiment, if a solution is unable to be identified within the database, the human expert may be notified. The human expert may then provide additional information to direct the care agent to provide a particular solution to the user. The care agent avatar will maintain its personalized mannerisms and behavior such that the user will be unaware that different people or computer processors are controlling what is being conveyed.

In an additional embodiment, the customer care agent may be utilized as a sales agent. The customer care agent may be directed to identify what service or item the user may wish to purchase. The customer care agent may employ similar tactics as described above to identify the service or item. Machine learning may also be used to develop a personalized sales approach which is effective for the user. For example, the customer care agent may learn particular techniques or tactics that are effective in selling a product to the user. As such, the customer care agent may employ those tactics in future sales interactions with the user. The customer care agent can suggest new products, convey product inventory availability, provide demonstrations, show basic setup and configuration, show simple repair steps, pricing options, and customer offers, ask questions to understand preferences and save in customer profile.

Finally at step 314, the personalized care agent is caused to communicate the solution or answer with the user. As previously explained, the avatar will be directed by either a computer processor or the human expert on how to interact with the user. The avatar will convey the messages that are identified previously and in a manner that is appropriate for each message. The care agent application may identify, using machine learning, what emotions the avatar should convey for different interactions with the user.

Many different arrangements of the various components depicted, as well as components not shown, are possible without departing from the scope of the claims below. Embodiments of our technology have been described with the intent to be illustrative rather than restrictive. Alternative embodiments will become apparent to readers of this disclosure after and because of reading it. Alternative means of implementing the aforementioned can be completed without departing from the scope of the claims below. Certain features and sub-combinations are of utility and may be employed without reference to other features and sub-combinations and are contemplated within the scope of the claims.

The invention claimed is:

1. A method comprising:
receiving, from a user device, a request to access a care agent application;
authenticating, by way of a server, a user associated with the user device;
causing, by way of the virtual care
application, a personalized care agent avatar to be displayed on the user device;
causing the personalized care agent avatar to interact with the user, by way of the user device, to identify a first user concern;
accessing a personalized care database associated with the user;
identifying a solution to the first user concern;
causing the personalized care agent avatar to communicate the solution to the user by way of the user device.

2. The method of claim 1, wherein the care agent application exists within a metaverse.

3. The method of claim 1, wherein the authentication of the user requires the user to provide a set of user credentials to the care agent application.

4. The method of claim 1, wherein the personalized care agent avatar is based at least in part on user identified preferences for the personalized care agent avatar.

5. The method of claim 4, wherein the user identified credentials include avatar language, avatar appearance, and avatar demeanor.

6. The method of claim 1, wherein causing the personalized care agent to interact with the user includes the personalized care agent using a scripted set of questions.

7. The method of claim 6, further comprising a human intervention to direct the causing the personalized care agent to interact with the user, wherein the human intervention occurs following an identification that the scripted set of questions does not identify the first user concern.

8. The method of claim 1, wherein personalized care database includes information about the user's preferences, payment information, account information, historical information, demographics, and geographical location.

9. The method of claim 1, further comprising using a machine learning algorithm to identify the first user concern.

10. The method of claim 7, wherein solution is identified by the human intervention.

11. One or more non-transitory computer-readable media having computer-executable instructions embodied thereon that, when executed, perform a method comprising:
receiving, from a user device, a request to access a care agent application;
authenticating, by way of a server, a user associated with the user device;
causing, by way of the virtual care
application, a personalized care agent avatar to be displayed on the user device;
causing the personalized care agent avatar to interact with the user, by way of the user device, to identify a first user concern;
accessing a personalized care database associated with the user;
identifying a solution to the first user concern;
causing the personalized care agent avatar to communicate the solution to the user by way of the user device.

12. The method of claim 11, wherein causing the personalized care agent to interact with the user includes the personalized care agent using a scripted set of questions.

13. The method of claim 12, further comprising a human intervention to direct the causing the personalized care agent to interact with the user, wherein the human intervention occurs following an identification that the scripted set of questions does not identify the first user concern.

14. The method of claim 11, wherein personalized care database includes information about the user's preferences, payment information, account information, historical information, demographics, and geographical location.

15. The method of claim 11, further comprising using a machine learning algorithm to identify the first user concern.

16. The method of claim 13, wherein solution is identified by the human intervention.

17. A system for using a mobile drone delivery device, the system comprising:
one or more processors; and
one or more computer storage hardware devices storing computer-usable instructions that, when used by the one or more processors, cause the one or more processors to:
receive, from a user device, a request to access a care agent application;
authenticate, by way of a server, a user associated with the user device;
cause, by way of the virtual care application, a personalized care agent avatar to be displayed on the user device;
cause the personalized care agent avatar to interact with the user, by way of the user device, to identify a first user concern;
access a personalized care database associated with the user;
identify a solution to the first user concern;
cause the personalized care agent avatar to communicate the solution to the user by way of the user device.

18. The method of claim 17, further comprising a human intervention to direct the causing the personalized care agent to interact with the user, wherein the human intervention occurs following an identification that the scripted set of questions does not identify the first user concern.

19. The method of claim 17, wherein personalized care database includes information about the user's preferences, payment information, account information, historical information, demographics, and geographical location.

20. The method of claim 17, further comprising using a machine learning algorithm to identify the first user concern.
